(19) United States
(12) Patent Application Publication
(10) Pub. No.: US 2009/0068189 A1
(43) Pub. Date: Mar. 12, 2009

(54) Monoclonal Antibody to Human TGF-Beta Induced Gene-H3 and Use Thereof

(75) Inventors: Eun-Hee Bae, Seoul (KR); In-San Kim, Daegu (KR); Dong-Sin Lee, Gyeonggi-do (KR); Hee-Jeong Yun, Seoul (KR)

Correspondence Address: BUCHANAN, INGERSOLL & ROONEY PC, POST OFFICE BOX 1404, ALEXANDRIA, VA, 22313-1404, US

(73) Assignee: REGEN BIOTECH, INC., Gyeonggi-do (KR)

(21) Appl. No.: 11/791,043

(22) PCT Filed: Nov. 18, 2005
(86) PCT No.: PCT/KR05/03918

§ 371 Date: May 18, 2007

(30) Foreign Application Priority Data
Nov. 19, 2004 (KR) 10-2004-0095292

(51) Int. Cl. A61K 39/395; C07K 16/18; C12N 5/06; A61K 35/12; A61P 31/00; G01N 33/566; G01N 33/53; C40B 30/04

(52) U.S. Cl. 424/141.1; 530/388.2; 530/387.9; 435/332; 424/93.21; 436/501; 435/7.92; 506/9

(57) ABSTRACT

The present invention relates to a monoclonal antibody to human βig-h3 and the use thereof. More particularly, it relates to a monoclonal antibody to the human βig-h3 protein, wherein the epitope of the monoclonal antibody is the H1 region of the fourth fas-1 domain of the human βig-h3 protein. The monoclonal antibody can specifically recognize the human βig-h3 protein in tissue, and so will be useful in diagnosing a disease associated with the increase or decrease of the βig-h3 protein. In addition, the monoclonal antibody has the effect of inhibiting the cell adhesion activity of the βig-h3 protein.

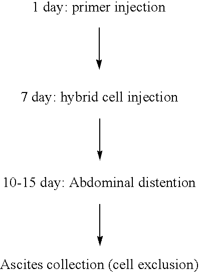

### FIELD OF THE INVENTION

This application claims priority to Korean Patent Application No. 2004-0095292, filed on Nov. 19, 2004, the contents of which are hereby incorporated by reference.

The present invention relates to a monoclonal antibody to human βig-h3 and the use thereof.

### BACKGROUND OF THE INVENTION

βig-h3 is an extracellular matrix protein whose expression is induced by TGF-β in various cells (Skonier et al., DNA Cell Biol. 11, 511-522, 1992). The βig-h3 protein was first isolated by Skonier et al. through the differential screening of a cDNA library constructed from human lung adenocarcinoma cells A549 treated with TGF-1 (Skonier J. et al., DNA Cell Biol., 11:511-522, 1992). The βig-h3 protein consists of 683 amino acids, and comprises an amino-terminal secretory sequence and a carboxy-terminal RGD ((Arg-Gly-Asp) motif that can serve as a ligand recognition site for several integrin receptors (Skonier, J. et al., DNA Cell Biol., 11:511, 1992). Also, the βig-h3 protein comprises four homologous internal repeat domains, which are homologous to similar motifs in the Drosophila fasciclin-I protein and thus are denoted fas-1 domains. The fas-1 domain has highly conserved sequences found in the secretory and membrane proteins of many organisms, including mammals, insects, sea urchins, plants, yeast, and bacteria (Kawamoto T., et al., Biochim. Biophys. Acta., 288-292, 1998). The fas-1 domain is comprised of about 110-140 amino acids, and particularly, comprises two highly conserved regions (H1 and H2) each consisting of about 10 amino acids.

It is known that the βig-h3 protein has a fibrillar structure and interacts with several extracellular matrix proteins, such as fibronectin and collagen (Kim J.-E., et al., Invest. Ophthalmol. Vis. Sci., 43:656-661, 2002). Also, it was reported that the βig-h3 protein is involved in cell growth and differentiation, wound healing and morphogenesis (Skonier J., et al., DNA Cell Biol., 13:571-584, 1994; Dieudonne S. C., et al., J. Cell. Biochem., 76:231-243, 1999; Kim J.-E., et al., J. Cell. Biochem., 77:169-178, 2000; Rawe I. M., et al., Invest. Opthalmol. Vis. Sci., 38:893-900, 1997; LeBaron R. G., et al., J. Invest. Dermatol., 104:844-849, 1995). In addition, big-h3 is known to mediate cell adhesion including corneal epithelial cells, chondrocytes, and fibroblasts (LeBaron R. G., et al., J. Invest. Dermatol., 104:844-849, 1995; Ohno S., et al., Biochim. Biophys. Acta, 1451: 196-205, 1999; Kim J.-E., et al., J. Biol. Chem., 275:30907-30915, 2000).

### DISCLOSURE OF THE INVENTION

The present inventors have developed a monoclonal antibody to the human βig-h3 protein and found that the monoclonal antibody specifically recognizes only the human βig-h3 protein without cross-reaction.

Therefore, it is an object of the present invention to provide a monoclonal antibody that specifically recognizes only the human βig-h3 protein, as well as the use thereof.

To achieve the above object, the present invention provides a monoclonal antibody specifically recognizing the human βig-h3 protein, wherein the epitope of the antibody is the H1 region of the fourth fas-1 domain of the human βig-h3 protein.

Another object of the present invention is to provide a hybridoma producing the monoclonal antibody.

Still another object of the present invention is to provide a method for preparing the monoclonal antibody.

Still another object of the present invention is to provide a kit for diagnosing a disease associated with an increase or decrease in the expression of βig-h3, the kit comprising the monoclonal antibody.

Still another object of the present invention is to provide a method for diagnosing a disease associated with an increase or a decrease in the expression of βig-h3 using the monoclonal antibody.

Still another object of the present invention is to provide a method for inhibiting the cell adhesion activity of βig-h3, the method comprising administering to a subject in need thereof an effective amount of the monoclonal antibody according to the present invention.

Yet another object of the present invention is to provide a method for inhibiting the metastasis of cancer, the method comprising administering to a subject in need thereof an effective amount of the monoclonal antibody according to the present invention.

### DEFINITIONS

Unless defined otherwise, all technical and scientific terms used herein have the same meanings as commonly understood by those skilled in the art to which the present invention pertains. The following references provide one of the skills having general definition for various terms used in the present invention: Singleton et al., DICTIONARY OF MICROBIOLOGY AND MOLECULAR BIOLOTY (2^{nd} ed. 1994); THE CAMBRIDGE DICTIONARY OF SCIENCE AND TECHNOLOGY (Walker ed., 1988); and Hale & Marham, THE HARPER COLLINS DICTIONARY OF BIOLOGY.

Also, the following definitions are provided to assist the reader in the practice of the present invention.

In the present invention, an antibody is generally obtained by introducing an antigen into an organism to induce a humoral immune response, inducing the production of an antibody to the antigen, and collecting blood from the organism. The antibody obtained by this method is called a polyclonal antibody. The polyclonal antibody is a mixture of antibodies secreted from plasma cells having different antibody genes. On the other hand, a single type of antibody, which reacts with only a single epitope and is prepared in single plasma cell having the same molecular structure, is referred to as a monoclonal antibody. Because the polyclonal antibody is a mixture of various antibodies, it can show undesired cross-reactivity with other antigens. However, a single type of antibody will recognize only a single specific epitope, and thus can provide very specific and precise results.

Hereinafter, the present invention will be described in detail.

The monoclonal antibody according to the present invention has an epitope corresponding to the H1 region of the fourth fas-1 domain of the human βig-h3 protein and is characterized by specifically recognizing the human βig-h3 protein. Preferably, the epitope of the inventive monoclonal antibody comprises an amino acid sequence shown in SEQ ID NO: 3. The human βig-h3 protein consists of 683 amino acids and comprises an RGD motif (Arg-Gly-Asp) serving as a ligand recognition site, and fas-1 domains consisting of four internal repeat domains. The full amino acid sequence of the human βig-h3 protein is shown in SEQ ID NO: 1. The fas-1 domains consist of 110-140 amino acids. More specifically, the four fas-1 domains of the βig-h3 protein consist of: the first fas-1 domain (D-I) corresponding to amino acids 133-236 of the βig-h3 protein; the second fas-1 domain (D-II) corresponding to amino acids 242-372 of the protein; the third fas-1 domain (D-III) corresponding to amino acids 373-501 of the protein; and the fourth fas-1 domain (D-IV; SEQ ID NO: 2) corresponding to amino acids 502-632 of the protein (see FIG. 5). The fourth fas-1 domain (D-IV) of the human βig-h3 protein includes two highly conserved regions (H1 and H2) each consisting of about 10 amino acids. The H1 region of βig-h3 D-IV region, which is the epitope of the inventive monoclonal antibody, has an amino acid sequence shown in SEQ ID NO: 3.

In one embodiment of the present invention, to determine the epitope of the inventive monoclonal antibody, a series of deletion mutants with a deletion in the H1 or H2 region of βig-h3 D-IV were prepared and analyzed by Western blot using the inventive monoclonal antibody. As a result, an antigen-antibody reaction did not occur only in the mutant with a deletion in H1 (see FIG. 8). These results suggest that the epitope of the inventive monoclonal antibody is the H1 region of the βig-h3 D-IV.

Meanwhile, the inventive monoclonal antibody is characterized by specifically recognizing only the human βig-h3 protein.

In one embodiment of the present invention, the inventive monoclonal antibody shows an antigen-antibody reaction with Mpt70 and Mpt83 proteins each having one fas-1 domain and produced by mycobacterium tuberculosis was examined. As a result, the inventive monoclonal antibody recognized βig-h3 D-IV did not recognize the Mpt70 and Mpt83 proteins (see FIG. 4).

Furthermore, the inventive monoclonal antibody specifically recognizes only the fourth fas-1 domain of the human βig-h3 protein and does not recognize the other fas-1 domains of the human βig-h3 protein.

In one embodiment of the present invention, whether the first fas-1 domain (D-I), second fas-1 domain (D-II) and third fas-1 domain (D-III) of the human βig-h3 protein are recognized by the inventive monoclonal antibody was examined by Western blot analysis. As a result, it could be found that the inventive monoclonal antibody specifically recognizes only the fourth fas-1 domain (D-IV) of the human βig-h3 protein (see FIG. 6).

Furthermore, the inventive monoclonal antibody is characterized in that it specifically recognizes only the human βig-h3 protein, and shows no cross-reaction with the mouse or rat βig-h3 protein.

In one embodiment of the present invention, a mouse chondrocyte culture fluid with induction of the mouse βig-h3 protein, a rat kidney cell culture fluid with induction of the rat βig-h3 protein, and a human lung adenocarcinoma cell culture fluid with induction of the human βig-h3 protein, were analyzed by Western blot using the inventive monoclonal antibody. As a result, it was shown that the inventive monoclonal antibody can recognize only the βig-h3 protein expressed in the human lung adenocarcinoma cell (see FIG. 11).

Also, in the present invention, whether the human βig-h3 protein expressed in tissues and cells is actually recognized by the inventive monoclonal antibody was examined by immunohistostaining using human kidney tissue and lung tissue (see FIG. 12). As a result, it could be found that the inventive monoclonal antibody can very specifically recognize the human βig-h3 protein expressed in tissues and cells.

The inventive monoclonal antibody can be prepared by a method comprising the steps of: (a) immunizing an animal using the fourth fas-1 domain of the βig-h3 protein as an immunogen; (b) fusing the spleen cell of the immunized animal with myeloma cell to produce a hybridoma; (c) selecting a positive clone producing a monoclonal antibody specifically recognizing human βig-h3; and (d) culturing the selected hybridoma and isolating an antibody from the hybridoma culture.

Also, the inventive monoclonal antibody can be prepared by injecting said hybridoma into the abdominal cavity of an animal, obtaining ascites from the animal at a given time after the injection, and isolating an antibody from the obtained ascites.

The fourth fas-1 domain of the human βig-h3 protein, which is used as an immunogen in the present invention, may be prepared by a genetic recombination method. For example, the fourth fas-1 domain of the human βig-h3 protein can be obtained by constructing cDNA using a known base sequence according to a conventional method known in the art, inserting the cDNA into an expression vector, expressing the cDNA in a host cell, and purifying the cDNA.

The protein expressed as described above can be isolated and purified from the fermentation or cell culture medium using conventional methods known in the art, for example, normal-phase or reverse-phase liquid chromatography using HPLC, FPLC, etc., affinity chromatography, size exclusion chromatography, immobilized metal chelate chromatography, and gel electrophoresis. Any person skilled in the art can readily select the most suitable isolation, and purification technique within the scope of the present invention. However, affinity chromatography can preferably be used.

Preferably, a recombinant protein consisting of four repeats of the fourth-1 domain of the human βig-h3 protein can be used as the immunogen in the present invention. Thus, the recombinant protein has a size similar to the original βig-h3 protein.

In one embodiment of the present invention, the cDNA of (βig-h3 D-IV as an antigen for preparing the monoclonal antibody was obtained by PCR amplification, and the cDNA was inserted into a vector to prepare a recombinant vector. For the purification of the recombinant protein, histidine residues were linked to the C-terminal region of the protein to make His-tag. The expression of the D-IV protein was induced using the recombinant vector, and the expressed protein was purified using Ni-NTA, and then, pure βig-h3 D-IV was purified by affinity chromatography using a polyclonal antibody. The purified βig-h3 D-IV was used as an immunogen for preparing the inventive monoclonal antibody (see Example 1).

To prepare the inventive monoclonal antibody, animals are immunized using the above-prepared immunogen as an antigen. More preferably, mouse and rat are used. The antigen is administered by intraperitoneal, intramuscular, intraocular or subcutaneous injection according to a conventional immunization method. If necessary, various techniques can be used to increase an immune reaction resulting from the protein and to develop increased antibody reactivity. For example, a complete or incomplete Freund's adjuvant can be used in the inventive antigen protein to increase the immunity of the protein. Although the immunity period is not specifically limited, the antigen is administered 2-10 times, and preferably 2-5 times, at an interval of several days to several weeks, and more preferably at an interval of 1-3 weeks. 1-10 days, preferably 2-5 days, after the final immunity, antibody-producing cells can be obtained from the animal. The antibody-producing cells include spleen cells, lymphocytes, thymocytes and peripheral blood cells. Preferably, the spleen cell can be used. When mouse is used, the antigen is administered in an amount of 1-100 μg/mouse, and preferably 25-50 μg/mouse.

The antibody-producing cells and myeloma cells obtained as described above are fused with each other according to a known method, for example, a method proposed by Koehler and Milstein. Examples of the myeloma cells which can be used mouse-derived cells, such as p3/x63-Ag8, p3-UI, NS-1, MPC-11, SP-2/0, F0, P3x63 Ag8. V653 and S194. In addition, rat-derived cell line, such as R-210, can be used.

From the hybridomas prepared as described above, a positive clone selectively recognizing the fourth fas-1 domain of the human βig-h3 protein is selected. The selection of the monoclone selectively recognizing the fourth fas-1 domain of the human βig-h3 protein can be performed using any immunochemical method known in the art. Examples of the immunochemical method include, but are not limited to, radioimmunoassay (RIA), enzyme-linked immunosorbent assay (ELISA), immunofluorescence, Western blotting and fluorescence activated cell sorting (FACS). Preferably, enzyme-linked immunosorbent assay (ELISA) is used.

In one embodiment of the present invention, hybridomas were prepared by immunizing a mouse using the inventive human βig-h3 D-IV recombinant protein as an antigen (see Example 2), isolating spleen cells from the immunized mouse and fusing the isolated spleen cells with myeloma cells (see Example 3). From the hybridomas, four positive clones selectively recognizing the human βig-h3 D-IV could be selected by ELISA (see Example 4).

The present inventor, from the above-selected four positive clones, selected 7A6a, which shows no antigen-antibody reaction with His-tag and has excellent cell viability and the highest antigen-antibody reactivity with βig-h3 D-IV.

The hybridoma 7A6a prepared in one embodiment of the present invention, which produces a monoclonal antibody to the human βig-h3 protein, was deposited under accession No. KCTC-10705BP on Oct. 11, 2004 with the Korean Collection for Type Cultures (KCTC), Korean Research Institute of Bioscience and Biotechnology, (52, Oun-dong, Yusong-ku, Taejon, Korea), which is an International Depository Authority under the Budapest Treaty. The deposit shall be maintained in viable condition at the KCTC during the entire term of the issued patent and shall be made available to any person or entity for non-commercial use without restriction, but in accordance with the provisions of the law governing the deposit. The hybridoma can be subcultured according to a conventional culture method and, if necessary, be frozen and stored. The hybridoma can be cultured by a conventional method, and either the culture medium can be obtained or the culture medium can be transplanted into the abdominal cavity of a mammal so as to obtain the ascites. The antibody in the culture medium or the ascites can be purified by conventional methods, such as salting out, ion exchange and gel permeation chromatography, and affinity column chromatography.

In another embodiment of the present invention, the above-obtained hybridoma was injected into the abdominal cavity of a mouse to produce ascites (see Example 9), and the ability of the mouse ascites to recognize the antibody was examined (see Example 10). As a result, it was found that the mouse ascites according to the present invention can recognize the human βig-h3 D-IV up to a concentration of 5 ng (see FIG. 10).

In still another aspect, the present invention provides a method for diagnosing a disease associated with an increase or a decrease in the expression of βig-h3 in a subject using the inventive the monoclonal antibody. This method may comprise the steps of: (a) contacting a test sample with the inventive monoclonal antibody; (b) detecting a product of immune reaction between the test sample and the monoclonal antibody to measure the expression level of βig-h3; (c) comparing the expression level of βig-h3 measured in the step (b) with the expression level of βig-h3 in a control sample.

As used herein, the term “test sample” refers to a biological sample obtained from subjects suspected of having said disease. For example, the term includes cells, tissue, blood and other liquid samples of biological origin, biopsy specimen, solid tissue samples, such as a tissue cultures, or cells derived therefrom and the progeny, but are not limited thereto. Also, sample refer to sample treated with reagents, and solubilized sample, or cultured cell, cell supernatant, cell lysate, and the like. The term “control sample” refers to a biological sample obtained from a normal subject.

In the step (a), the inventive antibody is preferably immobilized on a solid substrate. The antibody can be immobilized using various methods as disclosed in the literature (Antibodies: A Laboratory Manual, Harlow & Lane; Cold Spring Harbor, 1988). Suitable solid substrates include sticks, synthetic glass, agarose beads, cups, flat packs, those supporting by other solid supports, membrane attached them, or those coating by them. Other solid substrates include cell culture plates, ELISA plates, tubes, and polymeric membranes. The inventive antibody immobilized on the solid substrate can be treated with a test sample to contact with each other.

The term “product of immune reaction” in the step (b) refers to one produced by the antigen-antibody reaction between the βig-h3 protein in the test sample and the inventive antibody. The detection of the immune reaction product can be performed using any immunological assay method known in the art. Examples of the immunological assay method may include all assay methods which can measure the binding of the antigen to the inventive antibody. These assay methods are known in the art and include, for examples immunocytochemistry and immunohistochemistry, radioimmunoassay, enzyme-linked immunoabsorbent assay (ELISA), immunoprecipitation, immunoblotting, Farr assay, precipitin reaction, turbidimetry, immunodiffusion, counter-current electrophoresis, single radical immunodiffusion, protein chip, rapid assay, microarray, immunofluorescence and immunoadsorption.

The immunological assay can be performed using a suitable carrier used in all the known quantitative analysis method based on the principle of antigen-antibody bonding, a label capable of producing a detectable signal, a solubilizing agent and a cleaning agent. Suitable carriers include, but are not limited to, soluble carriers, for example, any one of physiologically acceptable buffers known in the art (e.g. PBS), or insoluble carriers, for example, polystyrene, polyethylene, polypropylene, polyester, polyacrylonitrile, fluorine resin, crosslinked dextran, polysaccharide, glass, metal, agarose and a combination thereof.

The expression level of βig-h3 in the test sample can be measured using a label capable of producing a detectable signal. Examples of the label capable of producing a detectable signal include enzymes, fluorescent substances, light-emitting substance and radioactive substances. The enzymes include peroxidase, alkaline phosphatase, β-D-galactosidase, glucose oxidase, maleate dehydrogenase, glucose-6-phosphodehydrogenase, invertase and the like. The fluorescent substances include fluorescein isothiocyanate, phycobilin protein, rhodamine, phycoerythrin, phycocyanin and orthophthalic aldehyde. The light-emitting substances include isolumino, lucigenin and the like. The radioactive substances include ¹³¹I, ¹⁴C, ³H, etc. But, in addition to the above-exemplified substances, any substance can be used as long as it is usable in immunological assay. The label can be linked to the inventive antibody or a secondary antibody capable of binding thereto. Any secondary antibody can be used without limitation as long as it is known in the art.

In the step (c), the expression level of βig-h3, measured in the step (b), is compared to the expression level of βig-h3 in the control sample so as to examine whether the expression level of βig-h3 was changed, thus diagnose a disease associated with an increase or a decrease in the expression level of βig-h3.

As used herein, the phrase “disease associated with an increase or a decrease in the expression level of βig-h3” refers to disease showing a characteristic in that the expression level of βig-h3 is increased or decreased compared to a normal level. Example of disease showing a characteristic in that the expression level of βig-h3 is increased compared to a normal level (an expression level, in the control sample) include, but are not limited to, kidney-diseases, liver diseases and rheumatoid diseases. The βig-h3 protein is characterized in that its expression is strongly induced by TGF-β that plays an important role in the pathological mechanism of kidney disease. More specifically, the concentration of βig-h3 in the urine of patients with diabetic kidney disease, patients before undergoing kidney transplantation surgery, and patients with renal failure, is shown to be higher than that in the urine of normal persons. Also, the concentration of βig-h3 in the tissue of patients with hepatitis, and in the synovial fluid of patients with degenerative arthritis and rheumatoid arthritis, is shown to be higher than that in normal person (Korean Patent Laid-Open Publication No. 2002-82421). Accordingly, the inventive monoclonal antibody will be highly useful in diagnosis the above-described diseases by measuring the concentration of βig-h3.

One embodiment of the present invention illustrates enzyme immunoassay (EIA) using the inventive monoclonal antibody. Namely, a direct sandwich assay was performed using the βig-h3 protein as a standard protein and using the inventive monoclonal antibody. As a result, the inventive monoclonal antibody showed a correlation coefficient of more than 0.98 in proportion to the concentration of the standard protein, indicating that it can detect βig-h3 in a very precise manner (see FIG. 13). Accordingly, the inventive monoclonal antibody can be used in, e.g. EIA, for diagnosis the above-described diseases.

In addition, the inventive monoclonal antibody can be used in inhibiting cell adhesion.

In one embodiment of the present invention, the human βig-h3 protein and the four fas-1 domains of the protein were attached to an ELISA plate and treated with the inventive monoclonal antibody and mouse fibroblasts so as to examine whether the inventive monoclonal antibody inhibited the adhesion between the proteins and the mouse fibroblasts. The result showed that the inventive monoclonal antibody inhibited the cell adhesion activity of the human βig-h3 protein and the fourth fas-1 domain of the protein (see FIG. 14).

In another embodiment of the present invention, the inhibition of cell adhesion activity of the human βig-h3 protein was examined at various treatment concentrations of the inventive monoclonal antibody. As a result, it could be found that the cell adhesion activity of the human βig-h3 protein was greatly inhibited even when it was treated with 1 μl of a monoclonal antibody produced from ascites (see FIG. 15).

Accordingly, the present invention provides a method for inhibiting the cell adhesion activity of βig-h3, comprising administering to a subject in need thereof an effective amount of the inventive monoclonal antibody.

As used herein, the term “cells” may encompass all cells whose adhesion are known to be mediated by βig-h3. Examples of the cells include, but are not limited to, corneal epithelial cell, chondrocytes and fibroblasts.

The term “subject” may include animals, and preferably mammals, including human beings. The term may also include cells, tissues, organs, etc., derived from the animals.

As used herein, the term “effective amount” refers to the amount of the monoclonal antibody, which shows a preventive or therapeutic effect when being administered to a subject. The dosage of the monoclonal antibody according to the present invention can be suitably selected depend on the age, body weight, sex, health condition and disease severity, as well as the administration route and subject.

Also, the monoclonal antibody according to the present invention can be administered alone or in combination with a pharmaceutically acceptable carrier.

As used herein, the term “pharmaceutically acceptable” refers to a physiologically acceptable composition which, when administered to human beings, will generally not cause allergic reactions, such as gastrointestinal disturbance, dizziness, and similar reactions. Examples of the pharmaceutically acceptable carrier comprise carriers for oral administration, such as lactose, starch, cellulose derivatives, magnesium stearate, and stearic acid, and carriers for parenteral administration, such as water, suitable oil, saline solution, aqueous glucose, and glycol. A stabilizer and a preservative can additionally be used in the present invention. Suitable stabilizers comprise antioxidants, such as sodium bisulphite, sodium sulphite and ascorbic acid. Suitable preservatives comprise benzalkonium chloride, methyl- or propyl-paraben, and chlorobutanol. Other pharmaceutically acceptable carriers can be found in the following literature: Remington's Pharmaceutical Sciences, 19th ed., Mack Publishing Company, Easton, Pa., 1995.

The monoclonal antibody according to the present invention can be formulated into a suitable form together with the above pharmaceutically acceptable carrier according to any method known in the art. Namely, the inventive pharmaceutical composition can be formulated into various parenteral or oral forms according to a conventional method. The parenteral dosage formulations typically include an injection formulation, such as an isotonic solution or a suspension. The injection formulation may be prepared using a suitable dispersing agent, wetting agent or suspending agent according to any method known in the art. For example, the injection formulation can be prepared by dissolving necessary components in saline or buffer. Also, the oral dosage formulations include, but are not limited to, powders, granules, tablets, pills and capsules.

The monoclonal antibody formulated as described above may be administered in an effective amount by various routes, including oral, transdermal, subcutaneous, intravenous and intramuscular routes.

Moreover, because the inventive monoclonal antibody can function to inhibit cell adhesion, it affects the invasiveness, adsorption and migration of cancer cell, which occur during the metastasis of primary cancer in test animals. Thus, it can be used for the investigation of metastasis ability of cell, the inhibition of metastasis of cancer tissue, or the inhibition of the adhesion of angiogenic epithelial cell, resulting from the formation of cancer tissue.

Accordingly, the present invention provides a method for inhibiting the metastasis of cancer, comprising administering to a subject in need thereof an effective amount of the inventive monoclonal antibody.

### BRIEF DESCRIPTION OF THE DRAWINGS

FIG. 1 is a schematic diagram showing the structures of human βig-h3 and recombinant protein FN115.

FIG. 2 shows the results of Western blot analysis for whether the inventive monoclonal antibody recognizes His-tag.

FIG. 3 is a schematic diagram showing the fas-1 domain structure of each of human βig-h3 D-IV, Mpt70 and Mpt83. Black part: a highly conserved region in the fas-1 domain; and gray part: a conserved region in the fas-1 domain.

FIG. 4 shows the results of Western blot analysis for whether the inventive monoclonal antibody recognizes Mpt70 and Mpt83.

FIG. 5 is a schematic diagram showing the fas-1 domains of the human βig-h3 protein.

FIG. 6 shows the results of Western blot analysis for whether the inventive monoclonal antibody recognizes the D-I, D-II, D-III and D-IV of the human βig-h3 protein.

FIG. 7 is a schematic diagram showing deletion mutants of human βig-h3 D-IV.

FIG. 8 shows the results of Western blot analysis for whether the inventive monoclonal antibody recognizes deletion mutants of human βig-h3 D-IV.

FIG. 9 shows a step of inducing the inventive monoclonal antibody by forming ascites in mice using the inventive hybridoma.

FIG. 10 shows the results of Western blot analysis for the recognition sensitivity of an antibody produced in the inventive mouse ascites.

FIG. 11 shows the results of Western blot analysis for the cross-reaction the inventive monoclonal antibody produced in ascites with a mouse and rat βig-h3 protein.

FIG. 12 illustrates photographs showing the results of immunohistostaining in human kidney tissue and lung tissue, conducted using the inventive monoclonal antibody.

Arrows: regions with detected βig-h3.

FIG. 13 shows the results of enzyme immunoassay (EIA) conducted using the inventive monoclonal antibody.

FIG. 14 shows the effects of the inventive monoclonal antibody on the inhibition of cell adhesion activity of the βig-h3 protein, as compared to those of a polyclonal antibody.

FIG. 15 shows the inhibition of cell adhesion activity of each of a βig-h3 protein and a D-IV domain at various treatment concentrations of the inventive monoclonal antibody.

### BEST MODE FOR CARRYING OUT THE INVENTION

Hereinafter, the present invention will be described in detail by examples. It is to be understood, however, that these examples are for illustrative purpose only and should not be construed as limiting the scope of the present invention.

### Example 1

### Preparation of Human βig-h3 D-IV Recombinant Protein

For an antigen producing the inventive monoclonal antibody, an expression vector comprising four repeats of the fourth fas-1 domain (D-IV) of the human βig-h3 protein was prepared according to a known method (Kim J.-E. et al., J. Biol. Chem., 275: 30907-30915, 2000; Korean Patent Registration No. 10-0382042).

<1-1> Preparation of Recombinant Vector

cDNA corresponding to the fourth fas-1 domain D-IV (SEQ ID NO: 2; amino acids 502-632) of the human βig-h3 protein was prepared in the following manner. A Asp718-BglII fragment with a partial deletion in the amino terminal region of βig-H3 cDNA was inserted into the EcoRV and EcoRI sites of expression vector pET-29β to prepare pHis-β-b. A D-IV domain region was amplified by PCR (polymerase chain reaction) using the prepared pHis-β-b as a template. The amplification product was cloned into the EcoRV and XhoI sites of vector pET-29b(+) (Novagen, USA) to prepare a pβig-h3 D-IV expression vector. The fragment of D-IV domain region was additionally inserted using restriction enzymes EcoRV and XhoI into the pβig-h3 D-IV expression vector, thus preparing a recombinant expression vector comprising four repeats of domain D-IV region, pβig-h3 D-IV4X. Herein, the recombinant protein consisting of four repeats of domain D-IV was prepared in order to allow the recombinant protein to have a size similar to the original βig-h3 protein so as to show similar property. Meanwhile, to purify the recombinant protein using Ni-NTA resin, 6 histidine residues were linked to the C-terminus of the cDNA fragment, thus making His-tag.

<1-2> Expression and Isolation of Recombinant Protein

The recombinant vector βig-h3 D-IV4X was transformed into E. coli BS21 (DE3), and then inoculated into LB medium (10 g/L tryptone, 5 g/L yeast extract, 5 g/L NaCl) comprising 50 μg/ml kanamycin. Then, the medium was incubated in an incubator at 37° C. until an absorbance of 0.5-0.6 at 600 nm was reached. Then, 1 mM IPTG (isopropyl-β-D-(−)-thiogalactopyranoside (Sigma) was added to the cultured medium, then cultured at 37° C. for 4 hours to induce the expression of the human βig-h3 D-IV recombinant protein. The resulting culture medium was centrifuged to obtain the bacterial cells. The obtained cells were suspended in lysis buffer (50 mM Tris-HCl (pH 8.0), 100 mM NaCl, 1 mM EDTA, 1% triton X-100, 1 mM PMSF, 0.5 mM DTT) and disrupted by sonicator. After repeating this procedure five times, the disrupted cells were centrifuged to obtain the supernatant.

The proteins expressed as inclusion bodies in the supernatant were added and bound to Ni-NTA resin (resin, Qiagen) for 2 hours. The mixture was placed into a column to which a binding buffer (20 mM Tris-Cl, 500 mM NaCl, 5 mM imidazole) was then added in the amount of five times the resin, and then the resulting solution was washed with the same amount of washing buffer (20 mM Tris-Cl, 500 mM NaCl, 20 mM imidazole). A recombinant protein bound to the column was eluted out with elution buffer comprising 300 mM imidazole.

<1-3> Identification and Quantification of Recombinant Protein

The purified protein obtained in Example <1-2> was measured for concentration by a Bradford protein assay (BioRad, Hercules, Calif.) and examined for protein size by SDS-PAGE. That is, the eluate obtained in Example <1-2> was allowed to react using a protein quantification system (Bio-Rad) and then measured for protein concentration at 595 nm using an automatic ELISA Reader. Also, the eluate was loaded on 12% SDS-polyacrylamide gel and subjected to SDS-PAGE at 80V. As a standard for comparing the molecular weight of the protein, a low molecular weight calibration kit (Pharmacia) was used. After the electrophoresis, the protein was stained with coomassie brilliant blue R-250 to determine the molecular weight of the protein.

As a result, the βig-h3 D-IV 4X had a molecular weight of about 68 kDa.

### Example 2

### Immunization of Mouse with Recombinant Protein

30 μg of the recombinant protein βig-h3 D-IV prepared in <Example I> was mixed well with the same amount of a complete Freund adjuvant (GIBCO BRL). 0.2 ml of the suspension was injected into the abdominal cavity of a Balb/C mouse (6-week old, female). 4 weeks after the first antigen inoculation, second antigen inoculation was performed, and then additional antigen inoculation was performed total four times at 2-week intervals. At this time, the same amount of the protein was mixed with an incomplete Freund adjuvant (GIBCO BRL) to prepare a suspension, and the suspension was injected into the abdominal cavity in an amount of 0.2 ml each time. After third injection into the abdominal cavity, blood was obtained from the tail vein of the mouse, and the serum obtained from the collected blood was measured for antibody titer. The antibody titer was measured by enzyme-linked immunosorbent assay (ELISA) using the recombinant protein βig-h3 D-IV as an antigen. For this purpose, 0.5 μg/ml of the recombinant protein as an antigen was added in an amount of 100 μl to each well of a 96-well plate and coated thereon at room temperature for 2 hours. The plate was washed one time with PBS, and 200 μl of the bovine serum albumin (BSA) was added to make a concentration of 1 mg/ml, and the plate was blocked at room temperature for 2 hours. The plate was washed one time with PBS to prepare an ELISA plate. The serum obtained from the mouse was diluted 1000-fold with PBS, and cultured at room temperature for 1 hour and then washed three times with PBS. Next, alkaline-phosphatase-labeled goat anti-mouse IgG (Sigma) as a secondary antibody was diluted 1:5000 and added in an amount of 100 μl to each well of the plate. The plate was incubated at room temperature for 1 hour and washed three times with phosphate buffer saline. Alkaline phosphatase substrate PNNP was added in an amount of 100 μl to each well of the plate so as to induce color development reaction. When the color development reaction appeared, the absorbance at 405 nm was measured. Herein, when the inverse of the dilution of a serum sample showing an absorbance higher than that of normal mouse serum by at least two times (more than 0.2) was more than 30,000, the serum sample was evaluated to have sufficient immunization. When the antibody titer was insufficient, the recombinant protein was again injected into the tail vein of the mouse.

### Example 3

### Preparation of Hybridomas

The spleen cells of the mouse immunized in Example 2 were isolated and fused with myeloma cell Sp210-Ag14 (ATCC CRL-1581) to prepare hybridomas.

After, the spleen cells of the immunized mouse were aseptically isolated, which only cell components were isolated from the spleen cells in a medium supplemented with DMEM (Gibco BRL, Dulbecco's Modified Eagle Medium). The isolated pure spleen cells were mixed with Sp210-Ag14 cells at a ratio of 10:1 while PEG (polyethylene glycol) solution was added to promote the fusion between the cells. The fused cells were added and mixed with HAT medium (Gibco BRL). 100 μl of the fused cells were added to an each well of a 96-well plate on which mouse feeder cells have previously been plated. Then, the plate was incubated at 37° C. for 7 days. After completion of the incubation, wells having colonies which have survived in the HAT medium to form hybridomas were selected.

### Example 4

### Selection of Positive Clones

The hybridomas prepared in <Example 3> were cultured and positive clones were selected from the cultured hybridomas using ELISA. The hybridomas of <Example 3> were inoculated into a 96-well culture dish in such a manner that 10 cells, 5 cells and 0.5 cells were placed in each well of the culture dish. At this time, the inoculated cells were cultured in HAT medium while the medium was replaced with fresh medium every 3 days. The culture supernatants of wells with formation of a single colony observed by a microscope were taken and ELISA was conducted in the same manner as in <Example 2> to select positive clones.

As a result, four positive clones were selected and named “7A6-a”, “9B2-a”, “9G12-b” and “10B2-b”, respectively.

### Example 5

### Examination on Whether Inventive Monoclonal Antibody Recognizes His-tag

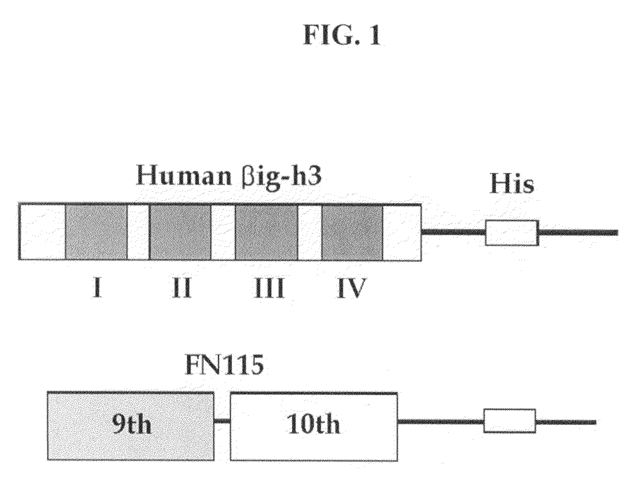

Whether four positive clones obtained in <Example 4> react with His-tag was examined. During the preparation of the human βig-h3 D-IV protein in <Example 1>, in order to the purification of the recombinant protein easy, His-tag was attached to the protein. Thus, whether the four positive clones react with the His-tag was analyzed by Western blot using a FN115 recombinant protein. The recombinant protein FN115 (33 kD) is prepared by inserting a pET29b(+) vector (Novagen, USA) a cDNA fragment comprising the ninth and tenth domains of fibronectin type III, transforming the vector into E. coli, inducing the expression of the protein and purifying the protein, and comprise His-tag attached thereto and has no similarity to βig-h3 D-IV (see FIG. 1) (Mardon, H. J., and Grant, K. E. FEBS Lett. 340, 197-201, 1994). Accordingly, if the four positive clones recognize the FN115 protein, they can be estimated to react with the His-tag.

First, each of the positive clones obtained in <Example 4> was inoculated in a medium supplemented with DMEM-low Glucose (Dulbeccos Modified Medium, Gibco BRL), 10% FBS (fetal bovine serum), 1% penicillin G and 1% streptomycin and was cultured in a 5% CO₂ incubator at 37° C.

The four positive clone culture fluids were analyzed by Western blot in the following manner. First, on the basis of the protein concentration measured by the Bradford protein assay, 50 ng of each of the human βig-h3 protein and the FN115 protein was electrophoresed on 12% SDS-polyacrylamide gel, and the gel was transferred to NC membranes. The membranes were incubated with 5% skim milk in TBS-T buffer (10 mM Tris-Cl, 150 mM NaCl, 0.05% Tween 20, pH 7.4) for 1 hour so as to block non-specific binding. Then, the NC membranes were immersed in each of the four positive clone culture fluids for 90 minutes, and then immunostained for 60 minutes in 5% skim milk comprising anti-mouse conjugated horseradish peroxidase at a concentration of 1:10000. The degree of immunostaining was observed with an enhanced chemiluminescence system (Amersham Pharmacia Bio-tech).

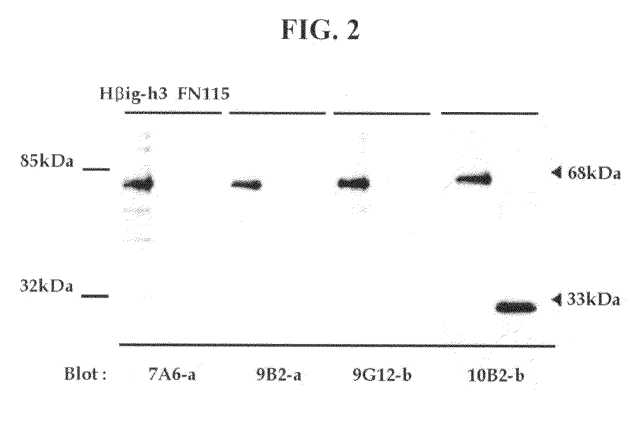

The test results showed that the four positive clones 7A6-a, 9B2-a, 9G12-b and 10B2-b all recognized the 68 kDa human βig-h3 protein. However, of the positive clones, 7A6-a, 9B2-a and 9G12-b did not recognize the FN115 protein, and only the positive clone 10B2-b recognized the 33-kDa FN115 protein (see FIG. 2).

Accordingly, it could be found that the positive clone 10B2-b has antigen-antibody reactivity with His-tag, and the remaining positive clones 7A6-a, 9B2-a and 9G12-b have no antigen-antibody reactivity with His-tag and specifically recognize only the human βig-h3 D-IV protein.

### Example 6

### Examination Whether Inventive Monoclonal Antibody Recognizes Mpt70 and Mpt83

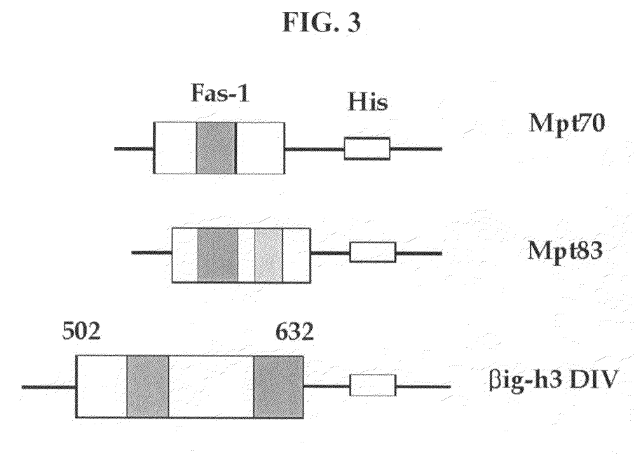

Mpt70 and Mpt83 produced in Mycobacterium tuberculosis belong to a huge gathering protein group of fas-1 (fasciclin 1 homologous domain) and each has one fas-1 domain (see FIG. 3). The positive clones obtained in <Example 4> have antigen-antibody reactivity with the fas-1 domain D-IV of the βig-h3 protein. Based on these particulars, whether the positive clones can also recognize the fas-1 domains of Mpt70 and Mpt83 was examined by Western blot analysis in the same manner as in <Example 5>. DNAs corresponding to the fas-1 domain (SEQ ID NO: 4) of Mpt70 (GenBank accession No. D37968) and the fas-1 domain (SEQ ID NO: 5) of Mpt83 (GenBank accession No. X94597), respectively, were amplified by PCR to obtain cDNA fragments. Each of the cDNA fragments was inserted into the BamHI and HindIII restriction enzyme sites of a pET29a vector to prepare expression vector. After each of the expression vectors was transformed into E. coli, which the expression of the proteins in E. coli was induced. The human βig-h3 D-IV protein prepared in <Example 1> and the above-prepared Mpt70 and Mpt83 proteins were separated on SDS-PAGE and attached on NC for analysis.

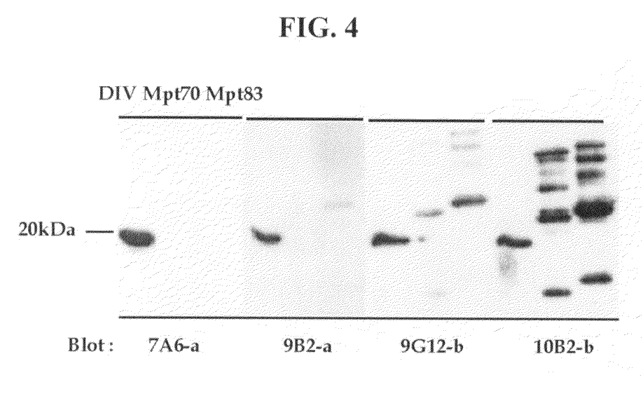

The analysis results showed that the positive clones 7A6-a, 9B2-a and 9G12-b recognized the human βig-h3 D-IV protein but did not recognize the Mpt70 and Mpt83 proteins. On the other hand, the positive clone 10B2-b recognized all the human βig-h3 D-IV, Mpt70 and Mpt83 proteins (see FIG. 4). It is thought that, because the Mpt70 and Mpt83 proteins have His-tag, the antigen-antibody reactivity of the positive clone 10B2-b with the Mpt70 and Mpt83 proteins is attributable to His-tag.

### Example 7

### Examination on Whether Inventive Monoclonal Antibody Recognizes D-I, D-II and D-III of Human βig-h3 Protein

It was examined whether the positive clones obtained in <Example 4> recognize not only the fas-1 domain D-IV of the human βig-h3 protein but also the D-I, D-II and D-III of the human βig-h3 protein.

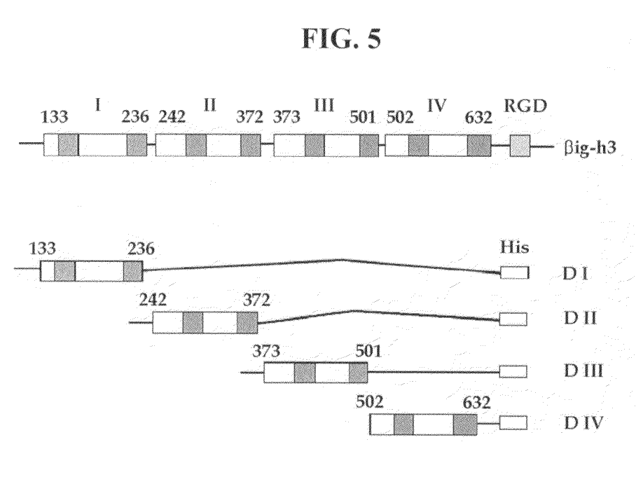

For this purpose, the D-I, D-II and D-III domains of the human βig-h3 protein were prepared in the same manner as in <Example 1>. To prepare these domains, cDNA fragments of human βig-h3 fas-1 domains (FIG. 5), which correspond to the first fas-1 domain D-I (amino acids 133-236), second fas-1 domain D-II (amino acids 242-372) and third fas-1 domain D-III (amino acids 373-501) of human βig-h3 (SEQ ID NO: 1), respectively, were amplified by PCR (polymerase chain reaction). Each of the amplification products was cloned into the EcoRV and XhoI sites of vector pET-29b(+), thus preparing expression vectors βig-h3 D-I, II and III. Also, in order to make the purification of recombinant proteins easy, 6 histidine residues were linked to the C-terminus of each of the fas-1 domains to make His-tag.

The expression of recombinant proteins was induced using the expression vectors in the same manner as in Example <1-2>, and the expressed proteins were purified, thus obtaining the human βig-h3 D-I, βig-h3 D-II and βig-h3 D-III proteins, respectively. The obtained proteins, together with human βig-h3 D-IV protein prepared in <Example 1>, were analyzed by Western blot in the same manner as in <Example 5>.

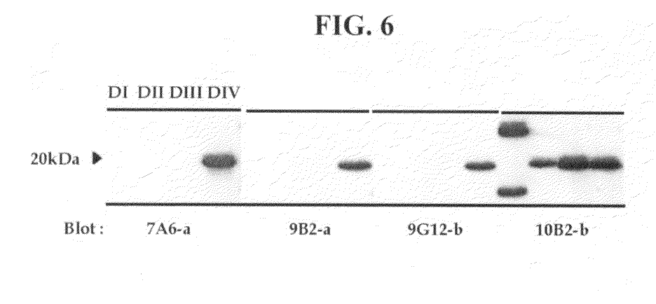

In the analysis results, the positive clones 7A6-a, 9B2-a and 9G12-b did not show antigen-antibody reactivity with the fas-1 domains except for the D-IV domain. On the other hand, the clone 10B2-b showed antigen-antibody reactivity with all the fas-1 domains (see FIG. 6). This is thought to be because the clone 10B2-b has antigen-antibody reactivity with His-tag.

From the above test results, it could be found that the positive clones 7A6-a, 9B2-a and 9G12-b are specific to the human βig-h3 D-IV domain.

Accordingly, among the four positive clones with the exception of the clone 10B2-b shown to have reactivity with His-tag, the clone 7A6-a was selected which has been found to have good cell viability and the highest antigen-antibody reactivity with the D-IV domain, based on the comparison between the results shown in <Example 5>, <Example 6> and <Example 7>. The selected clone 7A6-a was deposited under accession No. KCTC-10705BP on Oct. 11, 2004 with the Korean Collection for Type Cultures (KCTC), which is an International Depository Authority under the Budapest Treaty.

### Example 8

### Identification of Epitope of Inventive Monoclonal Antibody

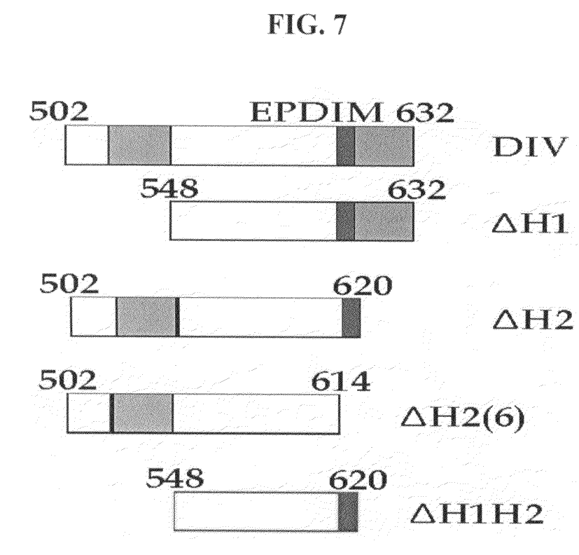

To identify the epitope of a monoclonal antibody produced by the inventive hybridoma 7A6-a, deletion mutants of human βig-h3. D-IV were prepared and analyzed by Western blot. As the deletion mutants of human βig-h3 D-IV, the following mutants were prepared: ΔH1 deleted in H1; ΔH2 deleted in H2; ΔH2(6) deleted in the peptide which has been more highly conserved in an evolutionally conserved H2 sequence and shows cell adhesion, diffusion and desorption activities; and ΔH1H2 deleted in all H1 and H2. DNA(ΔH1) corresponding to amino acids 548-632 of human βig-h3 (SEQ ID NO: 1), DNA(ΔH2) corresponding to amino acids 502-620 of βig-h3, DNA(ΔH2(6)) corresponding to amino acids 502-614 of βig-h3, and DNA(ΔH1H2) corresponding to amino acids 548-620 of βig-h3, were prepared by PCR amplification using the human βig-h3 D-IV cDNA prepared in Example <1-1> as a template (see FIG. 7).

The DNA fragment of each of the deletion mutants of human βig-h3 D-IV, obtained by the above PCR amplification, was cloned into a vector in the same manner as in Example <1-1> and expressed and purified in the same manner as in Example <1-2>.

The culture fluid of each of the ΔH1, ΔH2, ΔH2(6) and ΔH1H2 mutants, obtained as described above, with the positive clone 7A6-a, were analyzed by Western blot in the same manner as in <Example 5>. As a control group, the human βig-h3 D-IV prepared in <Example 1> was used.

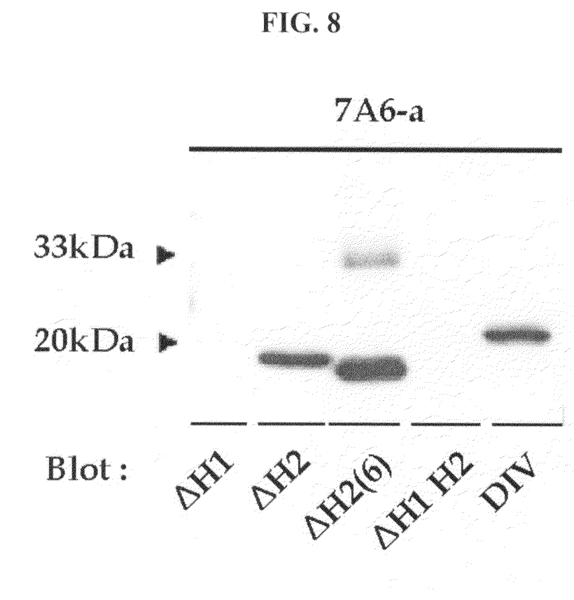

In the analysis results, only the mutant deleted in H1 did not show antigen-antibody reactivity with the inventive monoclonal antibody (see FIG. 8). This indicates that the epitope of the inventive 7A6-a is the H1 region (SEQ ID NO: 3).

### Example 9

### Induction of Formation of Mouse Ascites

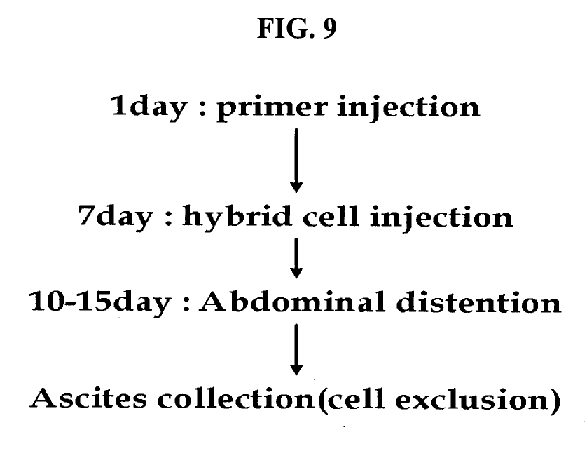

The hybridoma 7A6-a prepared in <Example 8> was injected into the abdominal cavity of a mouse to produce a high-concentration monoclonal antibody in the mouse ascites. For this purpose, 0.5 ml of 2,6,10,14-tetramethylpentadecane (pristane, Sigma T7640) was injected into the abdominal cavity of a BAL/C mouse (female) to make an environment where ascites can be formed. After 7 days, 5×10⁵ hybridoma cells were mixed with 1× phosphate-buffered saline and injected into the abdominal cavity of the mouse. 10 days after the injection, the mouse was anesthetized, and in this state, ascites was isolated from the abdominal cavity by a syringe and centrifuged at 3,000 rpm for 5 minutes to collect the supernatant (see FIG. 9). 0.02% sodium azaid was added to the collected supernatant which was then stored at −20° C.

### Example 10

### Examination of Ability of Mouse Ascites to Recognize Antibody

Whether the mouse ascites prepared in <Example 9> can recognize human βig-h3 D-IV was examined by Western blot analysis in the same manner as in <Example 5>. Herein, the human βig-h3 D-IV was used in concentrations of 100 ng, 50 ng, 10 ng, 5 ng and 1 ng.

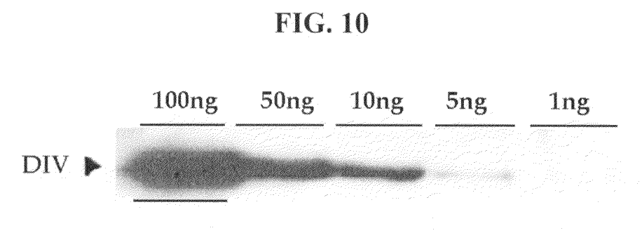

The test results showed that the monoclonal antibody produced in the ascites recognized the human βig-h3 D-IV up to a concentration of 5 ng (see FIG. 10).

### Example 11

### Examination of Cross-Reaction of Inventive Monoclonal Antibody with Mouse and rat βig-h3 Proteins

Mouse chondrocytes (ATDC5) inducing a mouse βig-h3 protein, and normal rat kidney cells (NRK, ATCC CRL-6509) inducing a rat βig-h3 protein, were cultured in conditions capable of inducing the βig-h3 proteins. Namely, the mouse chondrocytes and the rat kidney cells were proliferated in DMEM medium (comprising 4 mM L-glutamine, 1.5 g/L sodium bicarbonate, 4.5 g/L glucose, and 10% FBS), and then transferred and cultured in serum-free medium for 24 hours. Then, the culture fluids were collected. Also, human lung adenocarcinoma cells (H460, ATCC HTB-177) inducing the human βig-h3 protein were cultured in conditions capable of inducing the βig-h3 protein. Namely, the human lung adenocarcinoma cells were proliferated in RPMI 1640 medium (comprising 2 mM L-glutamine, 1.5 g/L sodium bicarbonate, 4.5 g/L glucose, 10 mM HEPES, 1.0 mM sodium pyruvate and 10% FBS) and then transferred and cultured in serum-free medium for 24 hours, and the culture fluid was collected. After the culture fluids were lyophilization, the concentration of the protein was analyzed based on bovine serum albumin standard according to the same Bradford assay (BioRad, Hercules, Calif.) as Example <1-3>. Then, whether the inventive monoclonal antibody recognizes the mouse and rat βig-h3 proteins was examined by Western blot analysis in the same manner as in <Example 5>. In the analysis, each of the culture fluids was used in an amount of 25 μl, and a polyclonal antibody to the mouse βig-h3 protein was used as a control group.

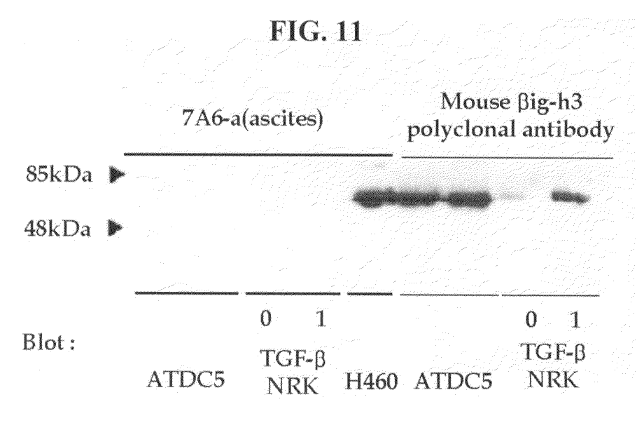

The test results showed that the monoclonal antibody to the inventive human βig-h3 protein could recognize the βig-h3 protein expressed in the human lung adenocarcinoma cells, but did not recognize the mouse and rat βig-h3 proteins. On the other hand, the polyclonal antibody to the mouse βig-h3 protein could recognize all the mouse and rat βig-h3 proteins (see FIG. 11).

Accordingly, it could be found that the inventive monoclonal antibody against the human βig-h3 protein shows no cross-reaction with the mouse and rat βig-h3 proteins.

### Example 12

### Detection of Big-h3 Protein in Human. Kidney Tissue and Lung Tissue Using the Inventive Monoclonal Antibody

In order to examine whether the inventive monoclonal antibody actually recognizes a βig-h3 protein expressed in tissue and cell, human kidney tissue and lung tissue were subjected to immunohistochemistry using the inventive monoclonal antibody.

For this purpose, kidney and lung tissues obtained from donors were fixed in 4% paraformaldehyde for 24 hours. The fixed tissues were dehydrated using Tissue-TEK (Sakura Finetek Japan co., Ltd.) and embedded in paraffin. The tissues were sectioned at 3 pm using a rotary microtome (Leica, Germany) and mounted on a slide. The paraffin of the tissue sections was removed with xylene, and in order to hydrate the tissues in biological conditions, the tissue sections were continuously hydrated in 99%, 96% and 70% ethanol solutions with gradually increasing water content. For indirect DAB immunostaining, the hydrated tissues were left in 3% H₂O₂ diluted with 100% methanol for 30 minutes to block peroxidase in the tissues. To activate antigen-antibody reactions in the tissues, the kidney tissue sections were heated in a microwave oven for 10 minutes. After cooling at room temperature for a long time, the tissue sections were incubated in 50 mM NH₄Cl for 30 minutes in order to prevent the non-specific binding of the antibody, and the slides were then treated with a solution (1% BSA, 0.05% saponin, and 0.2% gelatin in PBS) of preventing non-specific binding. Then, the inventive monoclonal antibody to the human βig-h3 protein was diluted at 1:500 with antibody dilution solution (0.1% BSA, 6.3% triton X-100 in PBS), and the sections were treated with the diluted antibody solution and allowed to react at 4° C. overnight. After completion of the reaction, the reaction solution was washed three times with washing solution (0.1% BSA, 0.05% saponin, 0.2% gelatin in PBS) for 10 minutes each time. Then, horseradish peroxidase-conjugated goat anti-mouse immunoglobulin (Santa Cruze Biotechnology) was diluted at 1:200 with antibody dilution solution, and tissue sections were treated with the diluted antibody solution. The resulting sections were washed in the same manner as described above, and DAB was spread on the tissue sections, and after about 5 minutes, it was observed that the reaction appeared brown. After completion of the reaction, the tissue sections were rapidly washed with PBS and stained with hematoxylin (Sigma). Finally, the tissue sections were continuously dehydrated in ethanol solutions (70%, 96% and 99%) with gradually increasing ethanol concentrations. The dehydrated tissues were coverslipped with Permount SP15-500 (Fisher Scientific). The histological patterns of βig-h3 stained on the tissue sections were observed with an optical microscope (Zeiss light microscope, Carl Zeiss, Oberkochem, Gerrnany).

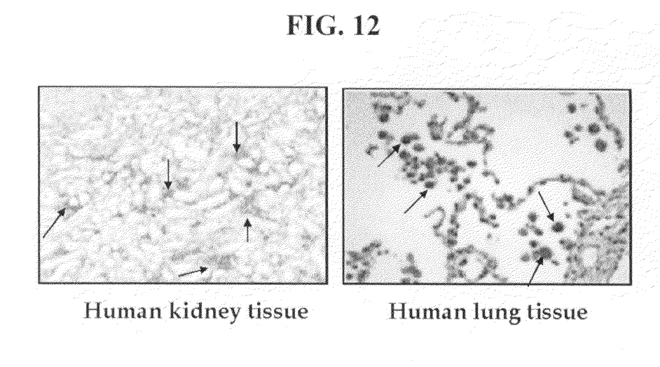

From the test results, it could be found that the inventive monoclonal antibody specifically recognizes the βig-h3 protein in the human kidney tissue and lung tissue (see FIG. 12).

### Example 13

### EIA (Enzyme Immunoassay) of Inventive Monoclonal Antibody

Whether the inventive monoclonal antibody can be used for the diagnosis of disease was examined using the direct sandwich method.

Using a βig-h3 protein with known concentration as a standard protein, tests were performed for the case where the inventive monoclonal antibody was conjugated with HRP and the case where the monoclonal antibody was used with a secondary antibody without conjugation. Also, a polyclonal antibody obtained by injecting a recombinant βig-h3 protein into a rabbit to induce immunization was used in the tests. First, a plate was coated with 100 μl (0.5 μg/ml) of the inventive non-conjugated monoclonal antibody and treated with blocking buffer to block non-specific reactions. Then, a recombinant βig-h3 protein with known concentration was added to the plate to induce an antigen-antibody reaction, and after a given time period, the reaction solution was removed. After washing, an antigen-antibody reaction was again induced using a rabbit-derived polyclonal antibody, and the amount of the βig-h3 protein was measured using the rabbit antibody reacted with the βig-h3 protein and an HRP-conjugated secondary antibody. According to the same manner as described above, a plate was coated with a rabbit polyclonal antibody, and the amount of the βig-h3 protein with known concentration was measured using an HRP-conjugated monoclonal antibody. The measurement results were graphed.

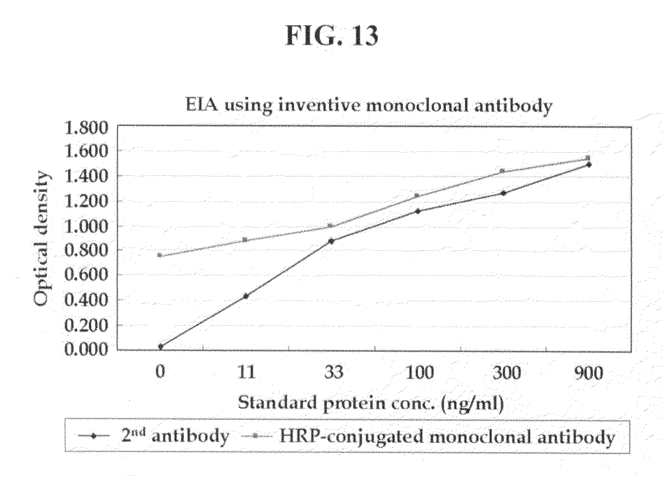

The test results showed that the case of coating with the monoclonal antibody had a little wider measurement range than the case of coating with the rabbit polyclonal antibody, and was lower in the background indicating the degree of non-specific reaction (see FIG. 13). However, both the two cases showed correlation coefficients of more than 0.98 in proportion to the concentration of the standard protein, suggesting that the inventive monoclonal antibody can be used in EIA.

### Example 14

### Examination on Whether Inventive Monoclonal Antibody Inhibits Cell Adhesion Activity of βig-h3 Protein

Whether the inventive monoclonal antibody inhibits the cell adhesion activity of the βig-h3 protein was examined.

A 96-well ELISA plate (Costar) was treated with each of pFN (purified human plasma fibronectin) (Sigma catalog #F 2006), βig-h3 D-I, βig-h3 D-II, βig-h3 D-III, βig-h3 D-IV and βig-h3 protein and allowed to react at 4° C. overnight so as to attach the proteins to the plate. As a control protein, bovine serum albumin was used. The plate having each of the proteins attached thereto was washed two times with phosphate buffer saline (PBS) and then treated with 2% bovine serum albumin and allowed to react for 1 hour so as to block non-specific reaction. The plate was washed two times with PBS, and then each of the proteins attached to the plate was treated with 50 μl (10 μg/ml) of the ascites of <Example 9> and allowed to react at 30° C. for 30 minutes. At this time, polyclonal antibodies to PBS, mouse IgG (Santa Cruz, USA) and human βig-h3 were added instead of the mouse ascites and compared to the case of addition of the mouse ascites. After completion of the reaction, 2.5×10⁴ mouse fibroblasts (NIH3T3) were added to the plate, and the adhesion of the fibroblasts was induced for a given time. Then, the plate was washed two times with 1×PBS, and 60 μl of 50 mM citrate buffer (pH 5.0) comprising 3.75 mM p-nitrophenyl-N-acetyl β-D-glycosamine (hexosaminidase substrate) and 25% triton X-100 was added to the plate and allowed to react at 37° C. for 1 hour. After completion of the reaction, 90 μl of 50 mM glycine comprising 5 mM EDTA was added to the plate so as to stop the enzyme activity. The enzymatic activity was measured at 450 nm using Model 550 microplate reader (Bio-Rad Laboratories, Inc., USA).

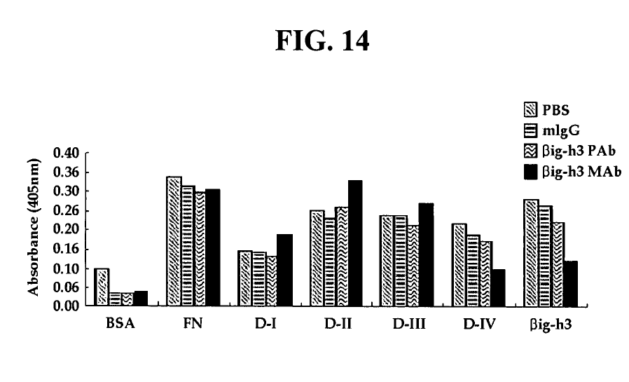

In the test result, the inhibition of cell adhesion activity by the inventive monoclonal antibody was shown only in the cases of using the human βig-h3 protein and the human βig-h3 D-IV protein (FIG. 14). Accordingly, it could be found that the inventive monoclonal antibody specifically recognizes the βig-h3 protein, particularly the fourth fas-1 domain thereof. The βig-h3 D-IV protein, thus inhibiting the adhesion activity of cells to the protein.

### Example 15

### Inhibition of Cell Adhesion Activity According to Treatment Concentration of Inventive Monoclonal Antibody

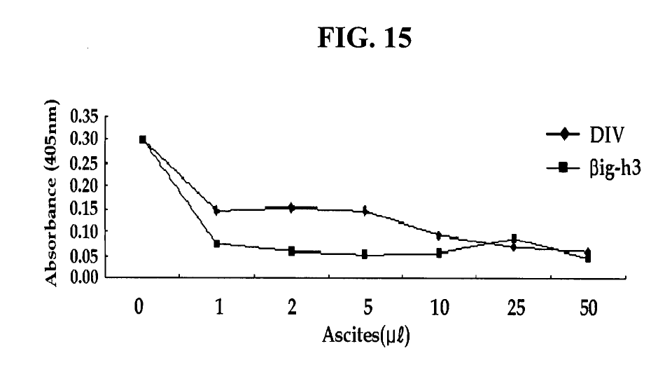

The inhibition of cell adhesion activity was measured in the same manner as in <Example 13>, except that, as proteins attached to the plate, only βig-h3 and βig-h3 D-IV were used, and the mouse ascites was used in amounts of 0, 1, 2, 5, 10, 25 and 50 μl. From the test results, it could be found that, even when the βig-h3 protein and the βig-h3 D-IV protein were treated with a low concentration of I Al of the inventive monoclonal antibody, the adhesion activities of the proteins to mouse fibroblasts were greatly reduced (see FIG. 15).

### INDUSTRIAL APPLICABILITY

The present inventive monoclonal antibody can specifically recognize the human βig-h3 protein in tissue, and so will be useful in diagnosing a disease associated with the increase or decrease of the βig-h3 protein. In addition, the monoclonal antibody has the effect of inhibiting the cell adhesion activity of the βig-h3 protein.

1. A monoclonal antibody specifically recognizing a human βig-h3 protein, wherein the epitope of the monoclonal antibody is the H1 region of the fourth fas-1 domain of the human βig-h3 protein.

2. The monoclonal antibody of claim 1, wherein the H1 region of the fourth fas-1 domain of the human βig-h3 protein has an amino acid sequence shown in SEQ ID NO: 3.

3. The monoclonal antibody of claim 1, which has no cross-reaction with a rat or mouse βig-h3 protein.

4. The monoclonal antibody of claim 1, which is produced by hybridoma 7A6-a (Accession No: KCTC 10705BP).

5. The monoclonal antibody of claim 1, which inhibits the cell adhesion activity of the human βig-h3 protein.

6. Hybridoma 7A6-a (Accession No: KCTC 10705BP) that produces the monoclonal antibody of claim 1.

7. A method for preparing a monoclonal antibody to human βig-h3, the method comprising the step of:
injecting the hybridoma of claim 6 into the abdominal cavity of a mouse;
obtaining ascites from the abdominal cavity; and
isolating the monoclonal antibody from the obtained ascites.

8. A kit for diagnosing a disease associated with, an increase or a decrease in βig-h3, the kit comprising the monoclonal antibody of claim 1.

9. The kit of claim 8, wherein the disease is selected from the group consisting of kidney disease, liver disease and rheumatoid disease.

10. A method for diagnosing a disease associated with an increase or a decrease in the expression level of βig-h3 in a subject, which comprises the steps of:
(a) contacting a test sample with the monoclonal antibody of claim 1;
(b) detecting a product of immune reaction between the test sample and the monoclonal antibody to measure the expression level of βig-h3; and
(c) comparing the expression level of βig-h3 measured in the step (b) with the expression level of βig-h3 in a control sample.

11. The method of claim 10, wherein the method for diagnosing is selected from the group consisting of immunocytochemistry and immunohistochemistry, radioimmunoassay, enzyme-linked immunoabsorbent assay (ELISA), immunoprecipitation, immunoblotting, Farr assay, precipitin reaction, turbidimetry, immunodiffusion, counter-current electrophoresis, single radical immunodiffusion, protein chip, rapid assay, microarray, immunofluorescence and immunosorbent assay.

12. The method of claim 10, wherein the disease is selected from the group consisting of kidney disease, liver disease and rheumatoid disease.

13. A method for inhibiting the cell adhesion activity of βig-h3, comprising administering to a subject in need thereof an effective amount of the monoclonal antibody of claim 1.

14. A method for inhibiting cancer metastasis, comprising administering to a subject in need thereof an effective amount of the monoclonal antibody of claim 1.


SEQUENCE LISTING (5 sequences)

SEQ ID NO: 1 - protein, 683 aa, Homo sapiens
  PEPTIDE (1)..(683): TGF-beta induced gene-h3
MALFVRLLALALALALGPAATLAGPAKSPYQLVLQHSRLRGRQHGPNVCAVQKVIGTNRKYFTNCKQWYQRKICGKSTVISYECCPGYEKVPGEKGCPAALPLSNLYETLGVVGSTTTQLYTDRTEKLRPEMEGPGSFTIFAPSNEAWASLPAEVLDSLVSNVNIELLNALRYHMVGRRVLTDELKHGMTLTSMYQNSNIQIHHYPNGIVTVNCARLLKADHHATNGVVHLIDKVISTITNNIQQIIEIEDTFETLRAAVAASGLNTMLEGNGQYTLLAPTNEAFEKIPSETLNRILGDPEALRDLLNNHILKSAMCAEAIVAGLSVETLEGTTLEVGCSGDMLTINGKAIISNKDILATNGVIHYIDELLIPDSAKTLFELAAESDVSTAIDLFRQAGLGNHLSGSERLTLLAPLNSVFKDGTPPIDAHTRNLLRNHIIKDQLASKYLYHGQTLETLGGKKLRVFVYRNSLCIENSCIAAHDKRGRYGTLFTMDRVLTPPMGTVMDVLKGDNRFSMLVAAIQSAGLTETLNREGVYTVFAPTNEAFRALPPRERSRLLGDAKELANILKYHIGDEILVSGGIGALVRLKSLQGDKLEVSLKNNVVSVNKEPVAEPDIMATNGVVHVITNVLQPPANRPQERGDELADSALEIFKQASAFSRASQRSVRLAPVYQKLLERMKH

SEQ ID NO: 2 - protein, 131 aa, Homo sapiens
  DOMAIN (1)..(131): fas-1 domain D-IV
MGTVMDVLKGDNRFSMLVAAIQSAGLTETLNREGVYTVFAPTNEAFRALPPRERSRLLGDAKELANILKYHIGDEILVSGGIGALVRLKSLQGDKLEVSLKNNVVSVNKEPVAEPDIMATNGVVHVITNVL

SEQ ID NO: 3 - protein, 46 aa, Homo sapiens
  DOMAIN (1)..(45): H1 of domain IV
MGTVMDVLKGDNRFSMLVAAIQSAGLTETLNREGVYTVFAPTNEAF

SEQ ID NO: 4 - protein, 96 aa, Mycobacterium tuberculosis
  DOMAIN (1)..(96): fas-1 domain of mpt70
TVFAPTNAAFSKLPASTIDELKTNSSLLTSILTYHVVAGQTSPANVVGTRQTLQGASVTVTGQGNSLKVGNADVVCGGVSTANATVYMIDSVLMPP

SEQ ID NO: 5 - protein, 96 aa, Mycobacterium tuberculosis
  DOMAIN (1)..(96): fas-1 domain of mpt83
TVFAPTNAAFDKLPAATIDQLKTDAKLLSSILTYHVIAGQASPSRIDGTHQTLQGADLTVIGARDDLMVNNAGLVCGGVHTANATVYMIDTVLMPP